(19) United States
(12) Patent Application Publication
(10) Pub. No.: US 2003/0051267 A1
(43) Pub. Date: Mar. 13, 2003

(54) Targeted disruption of kinase suppressor of RAS

(75) Inventors: John D. McNeish, Mystic, CT (US); Jeffrey L. Stock, North Stonington, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/143,122

(22) Filed: May 9, 2002

Related U.S. Application Data
(60) Provisional application No. 60/290,059, filed on May 10, 2001.

(51) Int. Cl. A01K 67/027; C12Q 1/68; C12N 5/06

(52) U.S. Cl. 800/18; 435/354; 435/6

(57) ABSTRACT

The invention features non-human mammals and animal cells that contain a targeted disruption of a kinase suppressor of Ras (KSR) gene.

This application claims priority, under 35 U.S.C. § 119(e), from U.S. provisional application 60/290,059, which was filed May 10, 2001.

### FIELD OF THE INVENTION

The invention features genetically-modified non human mammals and animal cells containing a functionally disrupted kinase suppressor of Ras (KSR) gene.

### BACKGROUND OF THE INVENTION

KSR participates in the Ras signaling pathway (Vojtek and Der, J. Biol. Chem. 273: 19925-28, 1998); it operates downstream of Ras but upstream of, or parallel to, Raf (Therrien et al., Cell 83: 879-88, 1995). One pathway regulated by Ras is the sequential activation of the cytoplasmic kinases Raf, MEK1, and extracellular ligand regulated (ERK) mitogen-activated protein (MAP) kinase. KSR regulation of Ras signaling may be mediated via modulating these kinases, as suggested by the KSR interaction with Raf, MEK1, and MAP kinase (Xing et al., Curr. Biol. 7: 294-300,1997; Therrien et al., Genes Dev. 10: 2684-95, 1996; Michaud et al., Proc. Natl. Acad. Sci. 94: 12792-96, 1997; Denouel-Galy et al., Curr. Biol. 8: 46-55, 1998; Yu et al., Curr. Biol. 8: 56-64; and Joneson et al., J. Biol. Chem. 273: 7743-48, 1998) and as suggested by KSR inhibition of MAP kinase activation (Joneson et al., supra; Bell et al., J. Biol. Chem. 274: 7982-86,1999).

One mode proposed for KSR action is that the protein acts as a molecular scaffold like the budding yeast protein ste5, which links protein kinases (Therrien et al., 1996, supra). However, the function of KSR in Ras signaling still remains to be resolved. Thus, there is a need for additional research tools, including KSR knockout mice, to further define the KSR function in Ras signaling and the therapeutic implications associated with modulating KSR activity.

### SUMMARY OF THE INVENTION

The present invention features genetically-modified non-human mammals and animal cells containing a disrupted kinase suppressor of Ras (KSR) gene. In one aspect, the invention features a genetically-modified, non-human mammal, wherein the modification results in a functionally disrupted KSR gene. The mammal is heterozygous or homozygous for the modification. Preferably, the mammal is a rodent, and, more preferably, the rodent is a mouse.

In a second aspect, the invention features a genetically-modified animal cell, wherein the modification comprises a functionally disrupted KSR gene. The cell is heterozygous or homozygous for the modification. Preferably, the cell is an embryonic stem (ES) cell or an ES-like cell, and the cell is human or murine.

Another aspect of the invention features a method of identifying a gene that demonstrates modified expression as a result of reduced KSR activity in an animal cell, comprising assessing the expression profile of an animal cell containing a genetic modification that disrupts a KSR gene, and comparing the profile to that from a wild type cell. Preferably, the cell is homozygous for a genetic modification that disrupts the KSR gene.

The present invention features genetically-modified non-human mammals and animal cells containing a disrupted kinase suppressor of Ras (KSR). Those skilled in the art will fully understand the terms used herein in the description and the appendant claims to describe the present invention. Nonetheless, unless otherwise provided herein, the following terms are as described immediately below.

A non-human mammal or an animal cell that is “genetically-modified” is heterozygous or homozygous for a modification that is introduced into the non-human mammal or animal cell, or into a progenitor non-human mammal or animal cell, by genetic engineering. The standard methods of genetic engineering that are available for introducing the modification include homologous recombination, viral vector gene trapping, irradiation, chemical mutagenesis, and the transgenic expression of a nucleotide sequence encoding antisense RNA alone or in combination with catalytic ribozymes. Preferred methods for genetic modification to disrupt a gene are those which modify an endogenous gene by inserting a “foreign nucleic acid sequence” into the gene locus, e.g., by homologous recombination or viral vector gene trapping. A “foreign nucleic acid sequence” is an exogenous sequence that is non-naturally occurring in the gene. This insertion of foreign DNA can occur within any region of the KSR gene, e.g., in an enhancer, promoter, regulator region, noncoding region, coding region, intron, or exon. The most preferred method of genetic engineering for gene disruption is homologous recombination, in which the foreign nucleic acid sequence is inserted in a targeted manner either alone or in combination with a deletion of a portion of the endogenous gene sequence.

By a KSR gene that is “functionally disrupted” is meant a KSR gene that is genetically modified such that the cellular activity of the KSR polypeptide encoded by the disrupted gene is decreased or eliminated in cells that normally express a wild type version of the KSR gene. When the genetic modification effectively eliminates all wild type copies of the KSR gene in a cell (e.g., the genetically-modified, non-human mammal or animal cell is homozygous for the KSR gene disruption or the only wild type copy of the KSR gene originally present is now disrupted), then the genetic modification results in a reduction in KSR polypeptide activity as compared to an appropriate control cell that expresses the wild type KSR gene. This reduction in KSR polypeptide activity results from either reduced KSR gene expression (i.e., KSR mRNA levels are effectively reduced and produce reduced levels of KSR polypeptide) and/or because the disrupted KSR gene encodes a mutated polypeptide with reduced function or stability as compared to a wild type KSR polypeptide. Preferably, the activity of KSR polypeptide in the genetically-modified, non-human mammal or animal cell is reduced to 50% or less of wild type levels, more preferably, to 25% or less, and, even more preferably, to 10% or less of wild type levels. Most preferably, the KSR gene disruption results in non-detectable KSR activity.

By a “genetically-modified, non-human mammal” containing a functionally disrupted KSR gene is meant a non-human mammal that is originally produced, for example, by creating a blastocyst or embryo carrying the desired genetic modification and then implanting the blastocyst or embryo in a foster mother for in utero development. The genetically-modified blastocyst or embryo can be made, in the case of mice, by implanting a genetically-modified embryonic stem (ES) cell into a mouse blastocyst or by aggregating ES cells with tetraploid embryos. Alternatively, various species of genetically-modified embryos can be obtained by nuclear transfer. In the case of nuclear transfer, the donor cell is a somatic cell or a pluripotent stem cell, and it is engineered to contain the desired genetic modification that functionally disrupts the KSR gene. The nucleus of this cell is then transferred into a fertilized or parthenogenetic oocyte that is enucleated; the resultant embryo is reconstituted and developed into a blastocyst. A genetically-modified blastocyst produced by either of the above methods is then implanted into a foster mother according to standard methods well known to those skilled in the art. A “genetically-modified, non-human mammal” includes all progeny of the non-human mammals created by the methods described above, provided that the progeny inherit at least one copy of the genetic modification that functionally disrupts the KSR gene. It is preferred that all somatic cells and germlne cells of the genetically-modified non-human mammal contain the modification. Preferred non-human mammals that are genetically-modified to contain a disrupted KSR gene include rodents, such as mice and rats, cats, dogs, rabbits, guinea pigs, hamsters, sheep, pigs, and ferrets.

By a “genetically-modified animal cell” containing a functionally disrupted KSR gene is meant an animal cell, including a human cell, created by genetic engineering to contain a functionally disrupted KSR gene, as well as daughter cells that inherit the disrupted KSR gene. These cells may be genetically-modified in culture according to any standard method known in the art. As an alternative to genetically modifying the cells in culture, non-human mammalian cells may also be isolated from a genetically-modified, non-human mammal that contains a KSR gene disruption. The animal cells of the invention may be obtained from primary cell or tissue preparations as well as culture-adapted, tumorigenic, or transformed cell lines. These cells and cell lines are derived, for example, from endothelial cells, epithelial cells, islets, neurons and other neural tissue-derived cells, mesothelial cells, osteocytes, lymphocytes, chondrocytes, hematopoietic cells, immune cells, cells of the major glands or organs (e.g., testicle, liver, lung, heart, stomach, pancreas, kidney, and skin), muscle cells (including cells from skeletal muscle, smooth muscle, and cardiac muscle), exocrine or endocrine cells, fibroblasts, and embryonic and other totipotent or pluripotent stem cells (e.g., ES cells, ES-like cells, and embryonic germlne (EG) cells, and other stem cells, such as progenitor cells and tissue-derived stem cells). The preferred genetically-modified cells are ES cells, more preferably, mouse or rat ES cells, and, most preferably, human ES cells.

By “reduced KSR activity” is meant a decrease in the activity of the KSR enzyme as a result of genetic manipulation of the KSR gene that causes a reduction in the level of KSR polypeptide in a cell, or as the result of administration of a pharmacological agent that inhibits KSR activity.

By an “ES cell” or an “ES-like cell” is meant a pluripotent stem cell derived from an embryo, from a primordial germ cell, or from a teratocarcinoma, that is capable of indefinite self renewal as well as differentiation into cell types that are representative of all three embryonic germ layers.

Other features and advantages of the invention will be apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art, and that are able to be ascertained without undue experimentation. Additional guidance with respect to making and using nucleic acids and polypeptides is found in standard textbooks of molecular biology, protein science, and immunology (see, e.g., Davis et al., Basic Methods in Molecular Biology, Elsevir Sciences Publishing, Inc., New York, N.Y.,1986; Hames et al., Nucleic Acid Hybridization, IL Press, 1985; Molecular Cloning, Sambrook et al., Current Protocols in Molecular Biology, Eds. Ausubel et al., John Wiley and Sons; Current Protocols in Human Genetics, Eds. Dracopoli et al., John Wiley and Sons; Current Protocols in Protein Science, Eds. John E. Coligan et al., John Wiley and Sons; and Current Protocols in Immunology, Eds. John E. Coligan et al., John Wiley and Sons). All publications mentioned herein are incorporated by reference in their entireties.

### DESCRIPTION OF THE FIGURES

FIG. 1 is a schematic depicting the strategy used for creating and verifying the identity of KSR knockout mice, including the KSR gene targeting vector, the location for homologous recombination of the vector in the endogenous murine KSR gene, and the positions of primers used to verify gene targeting.

FIG. 2 is a Southern analysis of G418/gancyclovir resistant ES cells following electroporation of the cells with the KSR targeting vector. ES cells heterozygous for the targeted disruption of the KSR gene with the targeting vector displayed two bands at 8.3 and 7.0 kb. ES cells that did not undergo homologous recombination and targeted disruption displayed only the 7.0 kb band.

FIG. 3 shows the results of polymerase chain reaction (PCR)-based genotyping of wild-type (+/+), heterozygote (+/−), and homozygote (−/−) mice with respect to the KSR allele.

FIG. 4 shows the results of reverse transcriptase (RT) PCR-based expression analysis which confirmed that the KSR knockout mice failed to express KSR.

### DETAILED DESCRIPTION

Genetically-Modified Non-human Mammals and Animal Cells Containing a Disrupted KSR Gene

1. Genetically-Modified Non-human Mammals and Animal Cells

The genetically-modified, non-human mammals and genetically-modified animal cells, including human cells, of the invention are heterozygous or homozygous for a modification that functionally disrupts the KSR gene. The animal cells may be derived by genetically engineering cells in culture, or, in the case of non-human mammalian cells, the cells may be isolated from genetically-modified, non-human mammals.

The KSR gene locus is functionally disrupted by one of the several techniques for genetic modification known in the art, including chemical mutagenesis (Rinchik, Trends in Genetics 7: 15-21, 1991, Russell, Environmental & Molecular Mutagenesis 23 (Suppl. 24) 23-29, 1994), irradiation (Russell, supra), transgenic expression of KSR gene antisense RNA, either alone or in combination with a catalytic RNA ribozyme sequence (Luyckx et al., Proc. Natl. Acad. Sci. 96: 12174-79, 1999; Sokol et al., Transgenic Research 5: 363-71, 1996; Efrat et al., Proc. Natl. Acad. Sci. USA 91: 2051-55,1994; Larsson et al., Nucleic Acids Research 22: 2242-48, 1994) and, as further discussed below, the disruption of the KSR gene by the insertion of a foreign nucleic acid sequence into the KSR gene locus. Preferably, the foreign sequence is inserted by homologous recombination or by the insertion of a viral vector. Most preferably, the method of KSR gene disruption is homologous recombination and includes a deletion of a portion of the endogenous KSR gene sequence.

The integration of the foreign sequence functionally disrupts the KSR gene through one or more of the following mechanisms: by interfering with the KSR gene transcription or translation process (e.g., by interfering with promoter recognition, or by introducing a transcription termination site or a translational stop codon into the KSR gene); or by distorting the KSR gene coding sequence such that it no longer encodes a KSR polypeptide with normal function (e.g., by inserting a foreign coding sequence into the KSR gene coding sequence, by introducing a frameshift mutation or amino acid(s) substitution, or, in the case of a double crossover event, by deleting a portion of the KSR gene coding sequence that is required for expression of a functional KSR protein).

To insert a foreign sequence into a KSR gene locus in the genome of a cell, the foreign DNA sequence is introduced into the cell according to a standard method known in the art such as electroporation, calcium-phosphate precipitation, retroviral infection, microinjection, biolistics, liposome transfection, DEAE-dextran transfection, or transferrinfection (see, e.g., Neumann et al., EMBO J. 1: 841-845, 1982; Potter et al., Proc. Natl. Acad. Sci USA 81: 7161-65, 1984; Chu et al., Nucleic Adds Res. 15: 1311-26, 1987; Thomas and Capecchi, Cell 51: 503-12, 1987; Baum et al., Biotechniques 17: 1058-62, 1994; Biewenga et al., J. Neuroscience Methods 71: 67-75, 1997; Zhang et al., Biotechniques 15: 868-72, 1993; Ray and Gage, Biotechniques 13: 598-603,1992; Lo, Mol. Cell. Biol. 3: 1803-14, 1983; Nickoloffet al., Mol. Biotech. 10: 93-101, 1998; Linney et al., Dev. Biol. (Orlando) 213: 207-16, 1999; Zimmer and Gruss, Nature 338: 150-153, 1989; and Robertson et al., Nature 323: 445-48,1986). The preferred method for introducing foreign DNA into a cell is electroporation.

2. Homologous Recombination

The method of homologous recombination targets the KSR gene for disruption by introducing a KSR gene targeting vector into a cell containing a KSR gene. The ability of the vector to target the KSR gene for disruption stems from using a nucleotide sequence in the vector that is homologous to the KSR gene. This homology region facilitates hybridization between the vector and the endogenous sequence of the KSR gene. Upon hybridization, the probability of a crossover event between the targeting vector and genomic sequences greatly increases. This crossover event results in the integration of the vector sequence into the KSR gene locus and the functional disruption of the KSR gene.

General principles regarding the construction of vectors used for targeting are reviewed in Bradley et al. (Biotechnol. 10: 534, 1992). Two different types of vector can be used to insert DNA by homologous recombination: an insertion vector or a replacement vector. An insertion vector is circular DNA which contains a region of KSR gene homology with a double stranded break. Following hybridization between the homology region and the endogenous KSR gene, a single crossover event at the double stranded break results in the insertion of the entire vector sequence into the endogenous gene at the site of crossover.

The more preferred vector to use for homologous recombination is a replacement vector, which is colinear rather than circular. Replacement vector integration into the KSR gene requires a double crossover event, i.e. crossing over at two sites of hybridization between the targeting vector and the KSR gene. This double crossover event results in the integration of a vector sequence that is sandwiched between the two sites of crossover into the KSR gene and the deletion of the corresponding endogenous KSR gene sequence that originally spanned between the two sites of crossover (see, e.g., Thomas and Capecchi et al., Cell 51: 503-12, 1987; Mansour et al., Nature 336: 348-52, 1988; Mansour et al., Proc. Natl. Acad. Sci. USA 87: 7688-7692, 1990; and Mansour, GATA 7: 219-227, 1990).

A region of homology in a targeting vector is generally at least 100 nucleotides in length. Most preferably, the homology region is at least 1-5 kilobases (kb) in length. Although there is no demonstrated minimum length or minimum degree of relatedness required for a homology region, targeting efficiency for homologous recombination generally corresponds with the length and the degree of relatedness between the targeting vector and the KSR gene locus. In the case where a replacement vector is used, and a portion of the endogenous KSR gene is deleted upon homologous recombination, an additional consideration is the size of the deleted portion of the endogenous KSR gene. If this portion of the endogenous KSR gene is greater than 1 kb in length, then a targeting cassette with regions of homology that are longer than 1 kb is recommended to enhance the efficiency of recombination. Further guidance regarding the selection and use of sequences effective for homologous recombination is described in the literature (see, e.g., Deng and Capecchi, Mol. Cell. Biol. 12: 3365-3371, 1992; Bollag et al., Annu. Rev. Genet. 23: 199-225, 1989; and Waldman and Liskay, Mol. Cell. Biol. 8: 5350-5357, 1988).

A wide variety of cloning vectors may be used as vector backbones in the construction of the KSR gene targeting vectors of the present invention, including pBluescript-related plasmids (e.g., Bluescript KS+11), pQE70, pQE60, pQE-9, pBS, pD10, phagescript, phiX174, pBK Phagemid, pNH8A, pNH16a, pNH18Z, pNH46A, ptrc99a, pKK223-3, pKK233-3, pDR540, and pRIT5 PWLNEO, pSV2CAT, pXT1, pSG (Stratagene), pSVK3, PBPV, PMSG, and pSVL, pBR322 and pBR322-based vectors, pMB9, pBR325, pKH47, pBR328, pHC79, phage Charon 28, pKB11, pKSV-10, pK19 related plasmids, pUC plasmids, and the pGEM series of plasmids. These vectors are available from a variety of commercial sources (e.g., Boehringer Mannheim Biochemicals, Indianapolis, Ind.; Qiagen, Valencia, Calif.; Stratagene, La Jolla, Calif.; Promega, Madison, Wis.; and New England Biolabs, Beverly, Mass.). However, any other vectors, e.g. plasmids, viruses, or parts thereof, may be used as long as they are replicable and viable in the desired host. The vector may also comprise sequences which enable it to replicate in the host whose genome is to be modified. The use of such a vector can expand the interaction period during which recombination can occur, increasing the efficiency of targeting (see Molecular Biology, ed. Ausubel et al, Unit 9.16, FIG. 9.16.1).

The specific host employed for propagating the targeting vectors of the present invention is not critical. Examples include E. coi K12 RR1 (Bolivar et al., Gene 2: 95, 1977), E coli K12 HB101 (ATCC No. 33694), E. coli MM21 (ATCC No. 336780), E. coli DH1 (ATCC No. 33849), E. coli strain DH5a, and E. coli STBL2. Alternatively, hosts such as C. cerevisiae or B. subtilis can be used. The above-mentioned hosts are available commercially (e.g., Stratagene, La Jolla, Calif.; and Life Technologies, Rockville, Md.).

To create the targeting vector, a KSR gene targeting construct is added to an above-described vector backbone. The KSR gene targeting constructs of the invention have at least one KSR gene homology region. To make the KSR gene homology regions, a KSR genomic or cDNA sequence is used as a basis for producing polymerase chain reaction (PCR) primers. These primers are used to amplify the desired region of the KSR sequence by high fidelity PCR amplification (Mattila et al., Nucleic Acids Res. 19: 4967, 1991; Eckert and Kunkel 1: 17, 1991; and U.S. Pat. No. 4,683,202). The genomic sequence is obtained from a genomic clone library or from a preparation of genomic DNA, preferably from the animal species that is to be targeted for KSR gene disruption. Examples of KSR cDNA sequences that can be used to make a KSR targeting vector include Genbank Accession No. U43585 (mouse) and U43586 (human).

Preferably, the targeting constructs of the invention also include an exogenous nucleotide sequence encoding a positive marker protein. The stable expression of a positive marker after vector integration confers an identifiable characteristic on the cell without compromising cell viability. Therefore, in the case of a replacement vector, the marker gene is positioned between two flanking homology regions so that it integrates into the KSR gene following the double crossover event.

It is preferred that the positive marker protein is a selectable protein; the stable expression of such a protein in a cell confers a selectable phenotypic characteristic, i.e., the characteristic enhances the survival of the cell under otherwise lethal conditions. Thus, by imposing the selectable condition, one can isolate cells that stably express the positive selectable marker-encoding vector sequence from other cells that have not successfully integrated the vector sequence on the basis of viability. Examples of positive selectable marker proteins (and their agents of selection) include neo (G418 or kanomycin), hyg (hygromycin), hisD (histidinol), gpt (xanthine), ble (bleomycin), and hprt (hypoxanthine) (see, e.g., Capecchi and Thomas, U.S. Pat. No. 5,464,764, and Capecchi, Science 244: 1288-92, 1989). Other positive markers that may also be used as an alternative to a selectable marker include reporter proteins such as β-galactosidase, firefly luciferase, or GFP (see, e.g., Current Protocols in Cytometry, Unit 9.5, and Current Protocols in Molecular Biology, Unit 9.6, John Wiley & Sons, New York, N.Y., 2000).

The above-described positive selection step does not distinguish between cells that have integrated the vector by targeted homologous recombination at the KSR gene locus versus random, non-homologous integration of vector sequence into any chromosomal position. Therefore, when using a replacement vector for homologous recombination, it is also preferred to include a nucleotide sequence encoding a negative selectable marker protein. Expression of a negative selectable marker causes a cell expressing the marker to lose viability when exposed to a certain agent (i.e., the marker protein becomes lethal to the cell under certain selectable conditions). Examples of negative selectable markers (and their agents of lethality) include herpes simplex virus thymidine kinase (gancyclovir or 1,2deoxy-2-fluoro-α-d-arabinofuransyl-5-iodouracil), Hprt (6-thioguanine or 6-thioxanthine), and diphtheria toxin, ricin toxin, and cytosine deaminase (5-fluorocytosine).

The nucleotide sequence encoding the negative selectable marker is positioned outside of the two homology regions of the replacement vector. Given this positioning, cells will only integrate and stably express the negative selectable marker if integration occurs by random, non-homologous recombination; homologous recombination between the KSR gene and the two regions of homology in the targeting construct excludes the sequence encoding the negative selectable marker from integration. Thus, by imposing the negative condition, cells that have integrated the targeting vector by random, non-homologous recombination lose viability.

The above-described combination of positive and negative selectable markers is preferred because a series of positive and negative selection steps can be designed to more efficiently select only those cells that have undergone vector integration by homologous recombination, and, therefore, have a potentially disrupted KSR gene. Further examples of positive-negative selection schemes, selectable markers, and targeting constructs are described, for example, in U.S. Pat. No. 5,464,764, WO 94/06908, and Valancius and Smithies, Mol. Cell. Biol. 11: 1402,1991.

In order for a marker protein to be stably expressed upon vector integration, the targeting vector may be designed so that the marker coding sequence is operably linked to the endogenous KSR gene promoter upon vector integration. Expression of the marker is then driven by the KSR gene promoter in cells that normally express the KSR gene. Alternatively, each marker in the targeting construct of the vector may contain its own promoter that drives expression independent of the KSR gene promoter. This latter scheme has the advantage of allowing for expression of markers in cells that do not typically express the KSR gene (Smith and Berg, Cold Spring Harbor Symp. Quant. Biol. 49: 171, 1984; Sedivy and Sharp, Proc. Natl. Acad. Sci. (USA) 86: 227, 1989; Thomas and Capecchi, Cell 51: 503, 1987).

Exogenous promoters that can be used to drive marker gene expression include cell-specific or stage-specific promoters, constitutive promoters, and inducible or regulatable promoters. Non-limiting examples of these promoters include the herpes simplex thymidine kinase promoter, cytomegalovirus (CMV) promoter/enhancer, SV40 promoters, PGK promoter, PMC1-neo, metallothionein promoter, adenovirus late promoter, vaccinia virus 7.5K promoter, avian beta globin promoter, histone promoters (e.g., mouse histone H3-614), beta actin promoter, neuron-specific enolase, muscle actin promoter, and the cauliflower mosaic virus 35S promoter (see generally, Sambrook et al., Molecular Cloning, Vols. I-Ill, Cold Spring Harbor Laboratory Press, Cold Spring Harbor, N.Y., 1989, and Current Protocols in Molecular Biology, John Wiley & Sons, New York, N.Y., 2000; Stratagene, La Jolla, Calif.).

To confirm whether cells have integrated the vector sequence into the targeted KSR gene locus, primers or genomic probes that are specific for the desired vector integration event can be used in combination with PCR or Southern blot analysis to identify the presence of the desired vector integration into the KSR gene locus (Erlich et al., Science 252: 1643-51, 1991; Zimmer and Gruss, Nature 338: 150, 1989; Mouellic et al., Proc. Natl. Acad. Sci. (USA) 87: 4712,1990; and Shesely et al., Proc. Natl. Acad. Sci. (USA) 88: 4294, 1991).

3. Gene Trapping

Another method available for inserting a foreign nucleic acid sequence into the KSR gene locus to functionally disrupt the KSR gene is gene trapping. This method takes advantage of the cellular machinery present in all mammalian cells that splices exons into mRNA to insert a gene trap vector coding sequence into a gene in a random fashion. Once inserted, the gene trap vector creates a mutation that may functionally disrupt the trapped KSR gene. In contrast to homologous recombination, this system for mutagenesis creates largely random mutations. Thus, to obtain a genetically-modified cell that contains a functionally disrupted KSR gene, cells containing this particular mutation must be identified and selected from a pool of cells that contain random mutations in a variety of genes.

Gene trapping systems and vectors have been described for use in genetically modifying murine cells and other cell types (see, e.g., Allen et al., Nature 333: 852-55, 1988; Bellen et al., Genes Dev. 3: 1288-1300, 1989; Bier et al., Genes Dev. 3: 1273-1287, 1989; Bonnerot et al., J. Virol. 66: 4982-91, 1992; Brenner et al., Proc. Nat. Acad. Sci. USA 86: 5517-21, 1989; Chang et al., Virology 193: 737-47, 1993; Friedrich and Soriano, Methods Enzymol. 225: 681-701,1993; Friedrich and Soriano, Genes Dev. 5: 1513-23,1991; Goff, Methods Enzymol. 152: 469-81, 1987; Gossler et al., Science 244: 463-65, 1989; Hope, Develop. 113: 399-408, 1991; Kerr et al., Cold Spring Harb. Symp. Quant. Biol. 2: 767-776, 1989; Reddy et al., J. Virol. 65: 1507-1515, 1991; Reddy et al., Proc. Natl. Acad. Sci. U.S.A. 89: 6721-25, 1992; Skarnes et al., Genes Dev. 6: 903-918,1992; von Melchner and Ruley, J. Virol. 63: 3227-3233,1989; and Yoshida et al., Transgen. Res. 4: 277-87, 1995).

Promoter trap, or 5′, vectors contain, in 5′ to 3′ order, a splice acceptor sequence followed by an exon, which is typically characterized by a translation initiation codon and open reading frame and/or an internal ribosome entry site. In general, these promoter trap vectors do not contain promoters or operably linked splice donor sequences. Consequently, after integration into the cellular genome of the host cell, the promoter trap vector sequence intercepts the normal splicing of the upstream gene and acts as a terminal exon. Expression of the vector coding sequence is dependent upon the vector integrating into an intron of the disrupted gene in the proper reading frame. In such a case, the cellular splicing machinery splices exons from the trapped gene upstream of the vector coding sequence (Zambrowicz et al., WO 99/50426).

An alternative method for producing an effect similar to the above-described promoter trap vector is a vector that incorporates a nested set of stop codons present in, or otherwise engineered into, the region between the splice acceptor of the promoter trap vector and the translation initiation codon or polyadenylation sequence. The coding sequence can also be engineered to contain an independent ribosome entry site (IRES) so that the coding sequence will be expressed in a manner largely independent of the site of integration within the host cell genome. Typically, but not necessarily, an IRES is used in conjunction with a nested set of stop codons.

Another type of gene trapping scheme uses a 3′ gene trap vector. This type of vector contains, in operative combination, a promoter region, which mediates expression of an adjoining coding sequence, the coding sequence, and a splice donor sequence that defines the 3′ end of the coding sequence exon. After integration into a host cell genome, the transcript expressed by the vector promoter is spliced to a splice acceptor sequence from the trapped gene that is located downstream of the integrated gene trap vector sequence. Thus, the integration of the vector results in the expression of a fusion transcript comprising the coding sequence of the 3′ gene trap cassette and any downstream cellular exons, including the terminal exon and its polyadenylation signal. When such vectors integrate into a gene, the cellular splicing machinery splices the vector coding sequence upstream of the 3′ exons of the trapped gene. One advantage of such vectors is that the expression of the 3′ gene trap vectors is driven by a promoter within the gene trap cassette and does not require integration into a gene that is normally expressed in the host cell (Zambrowicz et al., WO 99/50426). Examples of transcriptional promoters and enhancers that may be incorporated into the 3′ gene trap vector include those discussed above with respect to targeting vectors.

The viral vector backbone used as the structural component for the promoter or 3′ gene trap vector may be selected from a wide range of vectors that can be inserted into the genome of a target cell. Suitable backbone vectors include, but are not limited to, herpes simplex virus vectors, adenovirus vectors, adeno-associated virus vectors, retroviral vectors, lentiviral vectors, pseudorabies virus, alpha-herpes virus vectors, and the like. A thorough review of viral vectors, in particular, viral vectors suitable for modifying nonreplicating cells and how to use such vectors in conjunction with the expression of an exogenous polynucleotide sequence, can be found in Viral Vectors: Gene Therapy and Neuroscience Applications, Eds. Caplitt and Loewy, Academic Press, San Diego, 1995.

Preferably, retroviral vectors are used for gene trapping. These vectors can be used in conjunction with retroviral packaging cell lines such as those described in U.S. Pat. No. 5,449,614. Where non-murine mammalian cells are used as target cells for genetic modification, amphotropic or pantropic packaging cell lines can be used to package suitable vectors (Ory et al., Proc. Natl. Acad. Sci., USA 93: 11400-11406,1996). Representative retroviral vectors that can be adapted to create the presently described 3′ gene trap vectors are described, for example, in U.S. Pat. No. 5,521,076.

The gene trapping vectors may contain one or more of the positive marker genes discussed above with respect to targeting vectors used for homologous recombination. Similar to their use in targeting vectors, these positive markers are used in gene trapping vectors to identify and select cells that have integrated the vector into the cell genome. The marker gene may be engineered to contain an independent ribosome entry site (IRES) so that the marker will be expressed in a manner largely independent of the location in which the vector has integrated into the target cell genome.

Given that gene trap vectors will integrate into the genome of infected host cells in a fairly random manner, a genetically-modified cell having a disrupted KSR gene must be identified from a population of cells that have undergone random vector integration. Preferably, the genetic modifications in the population of cells are of sufficient randomness and frequency such that the population represents mutations in essentially every gene found in the cell's genome, making it likely that a cell with a disrupted KSR gene will be identified from the population (see Zambrowicz et al., WO 99/50426; Sands et al., WO 98/14614).

Individual mutant cell lines containing a disrupted KSR gene are identified in a population of mutated cells using, for example, reverse transcription and polymerase chain reaction (PCR) to identify a mutation in a KSR gene sequence. This process can be streamlined by pooling clones. For example, to find an individual clone containing a disrupted KSR gene, RT-PCR is performed using one primer anchored in the gene trap vector and the other primer located in the KSR gene sequence. A positive RT-PCR result indicates that the vector sequence is encoded in the KSR gene transcript, indicating that KSR gene has been disrupted by a gene trap integration event (see, e.g., Sands et al., WO 98/14614).

4. Temporal, Spatial, and Inducible KSR Gene Disruptions

In certain embodiments of the present invention, a functional disruption of the endogenous KSR gene occurs at specific developmental or cell cycle stages (temporal disruption) or in specific cell types (spatial disruption). In other embodiments, the KSR gene disruption is inducible when certain conditions are present. A recombinase excision system, such as a Cre-Lox system, may be used to activate or inactivate the KSR gene at a specific developmental stage, in a particular tissue or cell type, or under particular environmental conditions. Generally, methods utilizing Cre-Lox technology are carried out as described by Torres and Kuhn, Laboratory Protocols for Conditional Gene Targeting, Oxford University Press, 1997. Methodology similar to that described for the Cre-Lox system can also be employed utilizing the FLP-FRT system. Further guidance regarding the use of recombinase excision systems for conditionally disrupting genes by homologous recombination or viral insertion is provided, for example, in U.S. Pat. No. 5,626,159, U.S. Pat. No. 5,527,695, U.S. Pat. No. 5,434,066, WO 98/29533, Orban et al., Proc. Nat. Acad. Sci. USA 89: 6861-65, 1992; O'Gorman et al., Science 251: 1351-55, 1991; Sauer et al., Nucleic Acids Research 17: 147-61, 1989; Barinaga, Science 265: 26-28, 1994; and Akagi et al., Nucleic Acids Res. 25: 1766-73,1997. More than one recombinase system can be used to genetically modify a non-human mammal or animal cell.

When using homologous recombination to disrupt the KSR gene in a temporal, spatial, or inducible fashion, using a recombinase system such as the Cre-Lox system, a portion of the KSR gene coding region is replaced by a targeting construct comprising the KSR gene coding region flanked by loxP sites. Non-human mammals and animal cells carrying this genetic modification contain a functional, loxP-flanked KSR gene. The temporal, spatial, or inducible aspect of the KSR gene disruption is caused by the expression pattern of an additional transgene, a Cre recombinase transgene, that is expressed in the non-human mammal or animal cell under the control of the desired spatially-regulated, temporally-regulated, or inducible promoter, respectively. A Cre recombinase targets the loxP sites for recombination. Therefore, when Cre expression is activated, the LoxP sites undergo recombination to excise the sandwiched KSR gene coding sequence, resulting in a functional disruption of the KSR gene (Rajewski et al., J. Clin. Invest. 98: 600-03,1996; St.-Onge et al., Nucleic Acids Res. 24: 3875-77, 1996; Agah et al., J. Clin. Invest. 100: 169-79, 1997; Brocard et al., Proc. Natl. Acad. Sci. USA 94:14559-63,1997; Feil et al., Proc. Natl. Acad. Sci. USA 93:10887-90, 1996; and Kuhn et al., Science 269: 1427-29, 1995).

A cell containing both a Cre recombinase transgene and loxP-flanked KSR gene can be generated through standard transgenic techniques or, in the case of genetically-modified, non-human mammals, by crossing genetically-modified, non-human mammals wherein one parent contains a loxP flanked KSR gene and the other contains a Cre recombinase transgene under the control of the desired promoter. Further guidance regarding the use of recombinase systems and specific promoters to temporally, spatially, or conditionally disrupt the KSR gene is found, for example, in Sauer, Meth. Enz. 225: 890-900, 1993, Gu et al., Science 265: 103-06, 1994, Araki et al., J. Biochem. 122: 977-82,1997, Dymecki, Proc. Natl. Acad. Sci. 93: 6191-96, 1996, and Meyers et al., Nature Genetics 18: 136-41, 1998.

An inducible disruption of the KSR gene can also be achieved by using a tetracycline responsive binary system (Gossen and Bujard, Proc. Natl. Acad. Sci. USA 89: 5547-51, 1992). This system involves genetically modifying a cell to introduce a Tet promoter into the endogenous KSR gene regulatory element and a transgene expressing a tetracycline-controllable repressor (TetR). In such a cell, the administration of tetracycline activates the TetR which, in turn, inhibits KSR gene expression and, therefore, functionally disrupts the KSR gene (St.-Onge et al., Nucleic Acids Res. 24: 3875-77, 1996, U.S. Pat. No. 5,922,927).

The above-described systems for temporal, spatial, and inducible disruptions of the KSR gene can also be adopted when using gene trapping as the method of genetic modification, for example, as described, in WO 98/29533.

5. Creating Genetically-Modified, Non-human Mammals and Animal Cells

The above-described methods for genetic modification can be used to functionally disrupt a KSR gene in virtually any type of somatic or stem cell derived from an animal. Genetically-modified animal cells of the invention include, but are not limited to, mammalian cells, including human cells, and avian cells. These cells may be derived from genetically engineering any animal cell line, such as culture-adapted, tumorigenic, or transformed cell lines, or they may be isolated from a genetically-modified, non-human mammal carrying the desired KSR genetic modification.

The cells may be heterozygous or homozygous for the disrupted KSR gene. To obtain cells that are homozygous for the KSR gene disruption (KSR−/−), direct, sequential targeting of both alleles can be performed. This process can be facilitated by recycling a positive selectable marker. According to this scheme the nucleotide sequence encoding the positive selectable marker is removed following the disruption of one allele using the Cre-Lox P system. Thus, the same vector can be used in a subsequent round of targeting to disrupt the second KSR gene allele (Abuin and Bradley, Mol. Cell. Biol. 16: 1851-56,1996; Sedivy et al., T.I.G. 15: 88-90, 1999; Cruz et al., Proc. Natl. Acad. Sci. (USA) 88: 7170-74, 1991; Mortensen et al., Proc. Natl. Acad. Sci. (USA) 88: 7036-40, 1991; te Riele et al., Nature (London) 348: 649-651, 1990).

An alternative strategy for obtaining ES cells that are KSR−/− is the homogenotization of cells from a population of cells that is heterozygous for the KSR gene disruption (KSR+/−). The method uses a scheme in which KSR+/− targeted clones that express a selectable drug resistance marker are selected against a very high drug concentration; this selection favors cells that express two copies of the sequence encoding the drug resistance marker and are, therefore, homozygous for the KSR gene disruption (Mortensen et al., Mol. Cell. Biol. 12: 2391-95,1992). In addition, genetically-modified animal cells can be obtained from genetically-modified KSR−/−non-human mammals that are created by mating non-human mammals that are KSR+/− in germlne cells, as further discussed below.

Following the genetic modification of the desired cell or cell line, the KSR gene locus can be confirmed as the site of modification by PCR analysis according to standard PCR or Southern blotting methods known in the art (see, e.g., U.S. Pat. No. 4,683,202; and Erlich et al., Science 252: 1643, 1991). Further verification of the functional disruption of the KSR gene may also be made if KSR gene messenger RNA (mRNA) levels and/or KSR polypeptide levels are reduced in cells that normally express the KSR gene. Measures of KSR gene mRNA levels may be obtained by using reverse transcriptase mediated polymerase chain reaction (RT-PCR), Northern blot analysis, or in situ hybridization. The quantification of KSR polypeptide levels produced by the cells can be made, for example, by standard immunoassay methods known in the art. Such immunoassays include, but are not limited to, competitive and non-competitive assay systems using techniques such as RIAs (radioimmunoassays), ELISAs (enzyme-linked immunosorbent assays), “sandwich” immunoassays, immunoradiometric assays, gel diffusion precipitin reactions, immunodiffusion assays, in situ immunoassays (using, for example, colloidal gold, enzymatic, or radioisotope labels), Western blots, 2-dimensional gel analysis, precipitation reactions, immunofluorescence assays, protein A assays, and immunoelectrophoresis assays.

Preferred genetically-modified animal cells are embryonic stem (ES) cells and ES-like cells. These cells are derived from the preimplantation embryos and blastocysts of various species, such as mice (Evans et al., Nature 129:154-156,1981; Martin, Proc. Natl. Acad. Sci., USA, 78: 7634-7638, 1981), pigs and sheep (Notanianni et al., J. Reprod. Fert. Suppl., 43: 255-260,1991; Campbell et al., Nature 380: 64-68,1996) and primates, including humans (Thomson et al., U.S. Pat. No. 5,843,780, Thomson et al., Science 282: 1145-1147, 1995; and Thomson et al., Proc. Natl. Acad. Sci. USA 92: 7844-7848,1995).

These types of cells are pluripotent. That is, under proper conditions, they differentiate into a wide variety of cell types derived from all three embryonic germ layers: ectoderm, mesoderm and endoderm. Depending upon the culture conditions, a sample of ES cells can be cultured indefinitely as stem cells, allowed to differentiate into a wide variety of different cell types within a single sample, or directed to differentiate into a specific cell type, such as macrophage-like cells, neuronal cells, cardiomyocytes, chondrocytes, adipocytes, smooth muscle cells, endothelial cells, skeletal muscle cells, keratinocytes, and hematopoietic cells, such as eosinophils, mast cells, erythroid progenitor cells, or megakaryocytes. Directed differentiation is accomplished by including specific growth factors or matrix components in the culture conditions, as further described, for example, in Keller et al., Curr. Opin. Cell Biol. 7: 862-69, 1995, Li et al., Curr. Biol. 8: 971, 1998, Klug et al., J. Clin. Invest. 98: 216-24, 1996, Lieschke et al., Exp. Hematol. 23: 328-34, 1995, Yamane et al., Blood 90: 3516-23, 1997, and Hirashima et al., Blood 93: 1253-63, 1999.

The particular embryonic stem cell line that is used for genetic modification is not critical; exemplary murine ES cell lines include AB-1 (McMahon and Bradley, Cell 62:1073-85, 1990), E14 (Hooper et al., Nature 326: 292-95, 1987), D3 (Doetschman et al., J. Embryol. Exp. Morph. 87: 27-45, 1985), CCE (Robertson et al, Nature 323: 445-48, 1986), RW4 (Genome Systems, St. Louis, Mo.), and DBA/1lacJ (Roach et al., Exp. Cell Res. 221: 520-25, 1995). Genetically-modified murine ES cells may be used to generate genetically-modified mice, according to published procedures (Robertson, 1987, Teratocarcinomas and Embryonic Stem Cells: A Practical Approach, Ed. E. J. Robertson, Oxford: IRL Press, pp. 71-112, 1987; Zjilstra et al., Nature 342: 435-438, 1989; and Schwartzberg et al., Science 246: 799-803, 1989).

Following confirmation that the ES cells contain the desired functional disruption of the KSR gene, these ES cells are then injected into suitable blastocyst hosts for generation of chimeric mice according to methods known in the art (Capecchi, Trends Genet. 5: 70, 1989). The particular mouse blastocysts employed in the present invention are not critical. Examples of such blastocysts include those derived from C57BL6 mice, C57BL6 Albino mice, Swiss outbred mice, CFLP mice, and MFI mice. Alternatively ES cells may be sandwiched between tetraploid embryos in aggregation wells (Nagy et al., Proc. Natl. Acad. Sci. USA90: 8424-8428, 1993).

The blastocysts or embryos containing the genetically-modified ES cells are then implanted in pseudopregnant female mice and allowed to develop in utero (Hogan et al., Manipulating the Mouse Embryo: A Laboratory Manual, Cold Spring Harbor Laboratory press, Cold Spring Harbor, N.Y. 1988; and Teratocarcinomas and Embryonic Stem Cells: A Practical Approach, E. J. Robertson, ed., IRL Press, Washington, D.C., 1987). The offspring born to the foster mothers may be screened to identify those that are chimeric for the KSR gene disruption. Generally, such offspring contain some cells that are derived from the genetically-modified donor ES cell as well as other cells from the original blastocyst. In such circumstances, offspring may be screened initially for mosaic coat color, where a coat color selection strategy has been employed, to distinguish cells derived from the donor ES cell from the other cells of the blastocyst. Alternatively, DNA from tail tissue of the offspring can be used to identify mice containing the genetically-modified cells.

The mating of chimeric mice that contain the KSR gene disruption in germ line cells produces progeny that possess the KSR gene disruption in all germ line cells and somatic cells. Mice that are heterozygous for the KSR gene disruption can then be crossed to produce homozygotes (see, e.g., U.S. Pat. No. 5,557,032, and U.S. Pat. No. 5,532,158).

An alternative to the above-described ES cell technology for transferring a genetic modification from a cell to a whole animal is to use nuclear transfer. This method can be employed to make other genetically-modified, non-human mammals besides mice, for example, sheep (McCreath et al., Nature 29: 1066-69, 2000; Campbell et al., Nature 389: 64-66, 1996; and Schnieke et al., Science 278: 2130-33, 1997) and calves (Cibelli et al., Science 280: 1256-58, 1998). Briefly, somatic cells (e.g., fibroblasts) or pluripotent stem cells (e.g., ES-like cells) are selected as nuclear donors and are genetically-modified to contain a functional disruption of the KSR gene. When inserting a DNA vector into a somatic cell to mutate the KSR gene, it is preferred that a promoterless marker be used in the vector such that vector integration into the KSR gene results in expression of the marker under the control of the KSR gene promoter (Sedivy and Dutriaux, T.I.G. 15: 88-90, 1999; McCreath et al., Nature 29: 1066-69, 2000). Nuclei from donor cells which have the appropriate KSR gene disruption are then transferred to fertilized or parthenogenetic oocytes that are enucleated (Campbell et al., Nature 380: 64, 1996; Wilmut et al., Nature 385: 810, 1997). Embryos are reconstructed, cultured to develop into the morula/blastocyst stage, and transferred into foster mothers for full term in utero development.

The present invention also encompasses the progeny of the genetically-modified, non-human mammals and genetically-modified animal cells. While the progeny are heterozygous or homozygous for the genetic modification that functionally disrupts the KSR gene, they may not be genetically identical to the parent non-human mammals and animal cells due to mutations or environmental influences, besides that of the original genetic disruption of the KSR gene, that may occur in succeeding generations.

The cells from a non-human genetically modified animal can be isolated from tissue or organs using techniques known to those of skill in the art. In one embodiment, the genetically modified cells of the invention are immortalized. In accordance with this embodiment, cells can be immortalized by genetically engineering the telomerase gene, an oncogene, e.g., mos or v-src, or an apoptosis-inhibiting gene, e.g., bcl-2, into the cells. Alternatively, cells can be immortalized by fusion with a hybridization partner utilizing techniques known to one of skill in the art.

6. “Humanized” Non-human Mammals and Animal Cells

The genetically-modified non-human mammals and animal cells (non-human) of the invention containing a disrupted endogenous KSR gene can be further modified to express the human KSR sequence (referred to herein as “humanized”). A preferred method for humanizing cells involves replacing the endogenous KSR sequence with nucleic acid sequence encoding the human KSR sequence by homologous recombination. The vectors are similar to those traditionally used as targeting vectors with respect to the 5′ and 3′ homology arms and positive/negative selection schemes. However, the vectors also include sequence that, after recombination, either substitutes the human KSR coding sequence for the endogenous sequence, or effects base pair changes, exon substitutions, or codon substitutions that modify the endogenous sequence to encode the human KSR. Once homologous recombinants have been identified, it is possible to excise any selection-based sequences (e.g., neo) by using Cre or Flp-mediated site directed recombination (Dymecki, Proc. Natl. Acad. Sci. 93: 6191-96, 1996).

When substituting the human KSR sequence for the endogenous sequence, it is preferred that these changes are introduced directly downstream of the endogenous translation start site. This positioning preserves the endogenous temporal and spatial expression patterns of the KSR gene. The human sequence can be the full length human cDNA sequence with a polyA tail attached at the 3′ end for proper processing or the whole genomic sequence (Shiao et al., Transgenic Res. 8: 295-302, 1999). Further guidance regarding these methods of genetically modifying cells and non-human mammals to replace expression of an endogenous gene with its human counterpart is found, for example, in Sullivan et al., J. Biol. Chem. 272: 17972-80, 1997, Reaume et al., J. Biol. Chem. 271: 23380-88, 1996, and Scott et al., U.S. Pat. No. 5,777,194).

Another method for creating such “humanized” organisms is a two step process involving the disruption of the endogenous gene followed by the introduction of a transgene encoding the human sequence by pronuclear microinjection into the knock-out embryos.

7. Uses for the Genetically-Modified Non-human Mammals and Animal Cells

KSR function and therapeutic relevance can be elucidated by investigating the phenotype of KSR−/− and KSR+/− non-human mammals and animals cells of the invention. For example, the genetically-modified KSR^{-/-} non-human mammals and animal cells can be used to determine whether the KSR plays a role in causing or preventing symptoms or phenotypes to develop in certain models of disease. If a symptom or phenotype is different in a KSR-^{-/-} non-human mammal or animal cell as compared to a KSR^{+/+} or KSR ^{+/-} non-human mammal or animal cell, then the KSR plays a role in regulating functions associated with the symptom or phenotype.

In addition, under circumstances in which an agent has been identified as a KSR agonist or antagonist (e.g., the agent significantly modifies one or more of the KSR polypeptide activities when the agent is administered to a KSR^{+/+} or KSR^{+/-} non-human mammal or animal cell), the genetically-modified KSR^{-/-} animal cells of the invention are useful to characterize any other effects caused by the agent besides those known to result from the (ant)agonism of KSR (i.e., the non-human mammals and animal cells can be used as negative controls). For example, if the administration of the agent causes an effect in a KSR^{+/+} non-human mammal or animal cell that is not known to be associated with KSR polypeptide activity, then one can determine whether the agent exerts this other effect solely or primarily through modulation of KSR by administering the agent to a corresponding KSR^{-/-} non-human mammal or animal cell. If this other effect is absent, or is significantly reduced, in the KSR^{-/-} non-human mammal or animal cell, then the other effect is mediated, at least in part, by KSR. However, if the KSR^{-/-} non-human mammal or animal cell exhibits the other effect to a degree comparable to the KSR^{+/+} or KSR^{+/-} A non-human mammal or animal cell, then the other effect is mediated by a pathway that does not involve KSR signaling.

The genetically modified non-human mammals and animal cells of the invention can also be used to identify genes whose expression is upregulated in KSR^{-/+} or KSR^{-/-} non-human mammals or animal cells relative to their respective wild-type control. Techniques known to those of skill in the art can be used to identify such genes. For example, DNA assays can be used to identify genes whose expression is upregulated in KSR^{-/+} or KSR^{-/-} mice to compensate for a deficiency in KSR expression. DNA arrays are known to those of skill in the art. See, e.g., U.S. Pat. No. 5,965,352; Schena et al., Science 270:467-470, 1995; DeRisi et al., Nature Genetics 14:457-460, 1996; Shalon et al., Genome Res. 6:639-645, 1996; and Schena et al., Proc. Natl. Acad. Sci. (USA) 93:10539-11286,1995.

### EXAMPLES

1. Library Hybridization

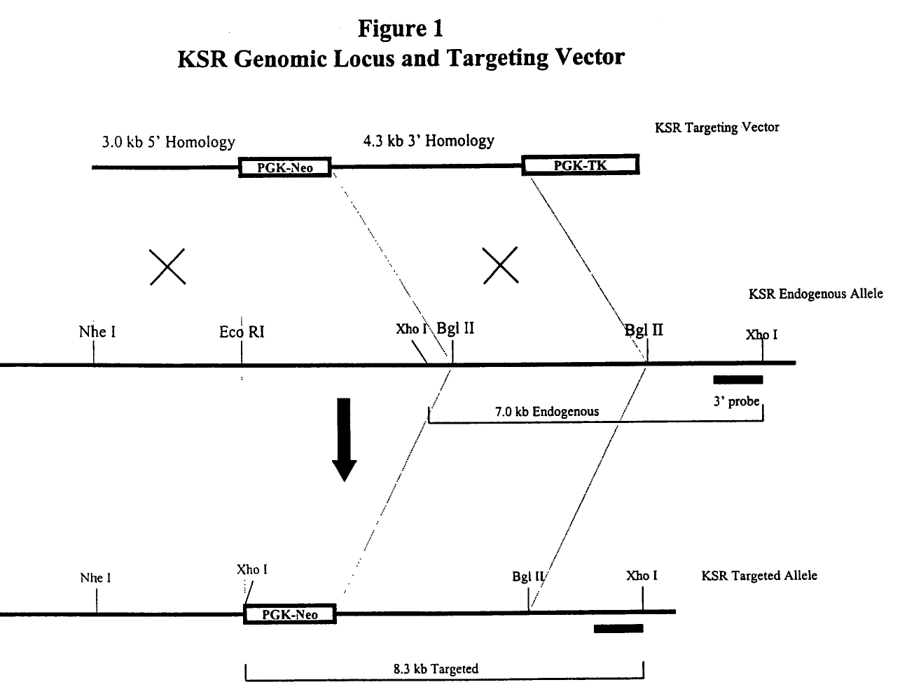

A targeting vector was constructed to delete a fragment of the KSR gene and replace it with the neomycin selectable marker gene (FIG. 1). A 1.5 kb genomic fragment was used to hybridize a genomic DBA/1 LacJ phage library (Stratagene, La Jolla, Calif.). This fragment contained KSR cDNA nucleotides 1086-1234 (as shown in Genbank Accession No. U43585) and a ˜1.3 kb intron. Three positive phage clones were isolated and genomic inserts were cloned into pBluescript SK+ (Stratagene, La Jolla, Calif.) using the NotI cloning site. These clones were restriction mapped and determined to contain 17 kb of the KSR genomic locus including at least 4 exons.

2. Targeting Vector Design

A 4.0 kb Bgl II fragment was isolated from one KSR genomic clone (#20-37) and subcloned into the BamHl site of a pJNS2 targeting vector backbone encoding Neo and TK as positive and negative selectable markers, respectively (Dombrowicz et al., Cell 75: 969-76, 1993). This Bgl II fragment represented the 3′ homology arm of the vector. For the 5′ homology arm, a 3.3 kb Nhel/EcoRI fragment was isolated from the same genomic clone (#20-37) and subcloned into pBluescript SK+ at the Xbal and EcoRi sites. This fragment was re-isolated from the pBluescript vector with a Notli/Xhol digestion and cloned into the Notl/Xhol site of the pJNS2 vector that already contained the 3′ homology arm at the Bgl II site. This completed targeting vector, containing both the 5′ and 3′ homology arms as well as Neo, was designed to replace 4.2 kb of genomic locus with the PGK-Neo selection cassette. This 4.2 kb fragment contains 2 exons encoding bp's 448-1024 of the KSR cDNA sequence, as reported in Genbank Accession No. U43585. Successful recombination of this targeting vector with the KSR locus resulted in the replacement of the endogenous KSR gene locus fragment with the neomycin selectable marker gene.

3. ES Cell Screening

The KSR targeting vector was linearized with Notl and electroporated into DBA/1 LacJ ES cells (Roach et al., Exp. Cell. Res. 221: 520-25, 1995). Pluripotent ES cells were maintained in culture on a mitomycin C treated primary embryonic fibroblast (PEF) feeder layer in stem cell medium (SCML) which consisted of knockout DMEM (Life Technologies, Inc., Gaithersburg, Md., Catalog No. LTI #10829-018) supplemented with 15% ES cell qualified fetal calf serum (Life Technologies, Inc., Catalog No. LTI #10439-024), 0.1 mM 2-mercaptoethanol (Sigma, St. Louis, Mo., #M-7522), 0.2 mM L-glutamine (Life Technologies, Inc., Catalog No. LTI #25030-081), 0.1 mM MEM non-essential amino acids (Life Technologies, Inc., Catalog No. LTI # 11140-050), 1000 units/ml recombinant murine leukemia inhibitory factor (Chemicon International Inc., Temecula, Calif., Catalog No. ESG-1107) and penicillin/streptomycin (Life Technologies, Inc., Catalog No. LTI #15140-122).

Electroporation of 1×10⁷ cells in SCML and 25 μg linearized targeting vector was carried out using a BTX Electro Cell Manipulator 600 (BTX, Inc., San Diego, Calif.) at a voltage of 240 V, a capacitance of 50 μF and a resistance of 360 Ohms. Positive/negative selection began 24 hours after electroporation in SCML which contained 200 μand 2 μM gancyclovir. Resistant colonies were picked with a micropipette following 8-12 days of selection. Expansion and screening of resistant ES cell colonies was performed as described in Mohn and Koller (Mohn, DNA Cloning 4 (ed. Hames), 143-184, Oxford University Press, New York, 1995).

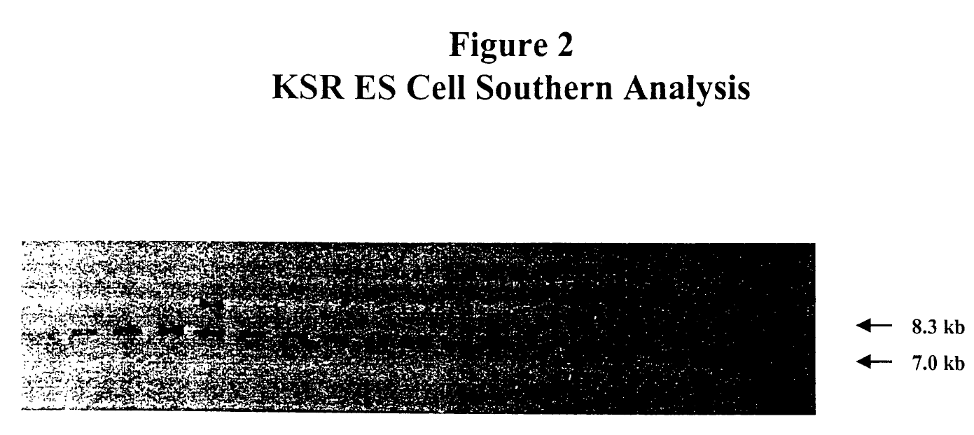

DNA was isolated from 95 ES cell clones which survived G418 and gancyclovir selection. A 1.0 kb Xhol/EcoRi fragment of the KSR gene downstream of the 3′ homology arm position, and, therefore, unaffected by homologous recombination, was used as a probe for Southern blot screening of the ES cell DNA (FIG. 1). The ES cell DNA was digested with Xhol restriction endonuclease and electrophoresed on a 0.7 % agarose gel (Biowhittaker Molecular Applications, Rockland, Me.). DNA from the gels was transferred to Hybond N+ (Amersham, Piscataway, NJ) nylon membrane for Southern analysis. The 1.0 kb probe recognized a 7.0 kb endogenous Xhol fragment and a 8.3 kb targeted fragment (FIG. 1 and FIG. 2). A total of 8 targeted clones were identified from the 95 screened using the 1.0 kb 3′ probe.

Targeting on the 5′ end of the targeting vector was confirmed for all clones using a KSR specific oligo upstream of the 5′ homology arm position (KSR 2037-418), and an oligo specific for the neomycin gene (Neo-257R). This oligo set amplifies a ˜4.0 kb fragment only if the targeting vector has recombined into the KSR endogenous locus.

| | |
|---|---|
| KSR 2037-418 | |
| 5′ CCTCCTTCTTGGCTCAATCTTTCC 3′ | (SEQ ID NO:1) |
| Neo-257R | |
| 5′ TGCTACTTCCATTTGTCACGTCC 3′ | (SEQ ID NO:2) |

4. Generation of Knockout Mice

Preparation of cells for injection into blastocysts was performed as described in Mohn and Koller (Mohn, DNA Cloning 4 (ed. Hames), 143-184 Oxford University Press, New York, 1995). Ten to fifteen targeted ES cells were introduced into the blastocoel of C57BL/6 embryos (Jackson Laboratories, Bar Harbor, Me.) and the embryos were allowed to continue development by reintroducing them into pseudopregnant foster mothers. Chimeric animals were identified for 3 of the 7 clones used for injections; male chimeric animals were backcrossed with DBA/1LacJ wild-type females (Jackson Laboratories) to generate germline F1 heterozygote animals inbred for the DBA/1LacJ strain.

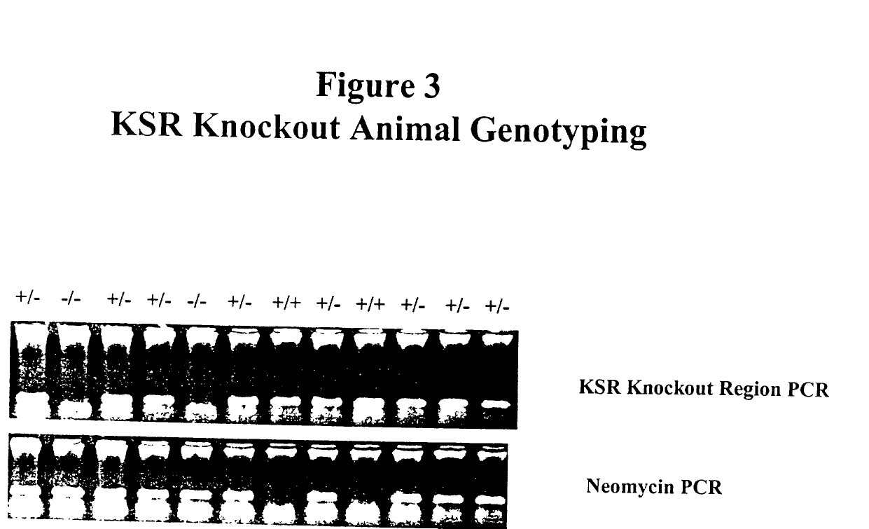

Homozygotes were generated by mating heterozygotes, and normal Mendelian ratios were observed. Wild-type, heterozygote, and homozygote animals were identified using a PCR-based genotyping method (FIG. 3). The method involved 2 PCR reactions, one specific for the KSR targeted allele and the second specific for the endogenous KSR allele. The oligo set used to PCR the targeted allele was specific for Neo and produced a 190 bp fragment.

| | |
|---|---|
| Neo-833F | |
| 5′ GCAGGATCTCCTGTCATCTCACC 3′ | (SEQ ID NO:3) |
| Neo-1023R | |
| 5′ GATGCTCTTCGTCCAGATCATCC 3′ | (SEQ ID NO:4) |

The oligo set used to PCR the KSR endogenous allele was specific to the knockout region and produced a 125 bp fragment.

| | | |
|---|---|---|
| KSR-883F | 5′ CAAGCTGAAGCCACCAAGG 3′ | (SEQ ID NO:5) |
| KSR-1008R | 5′ ACGTCGTCGATTCGGTTTCC 3′ | (SEQ ID NO:6) |

5. Expression Analysis

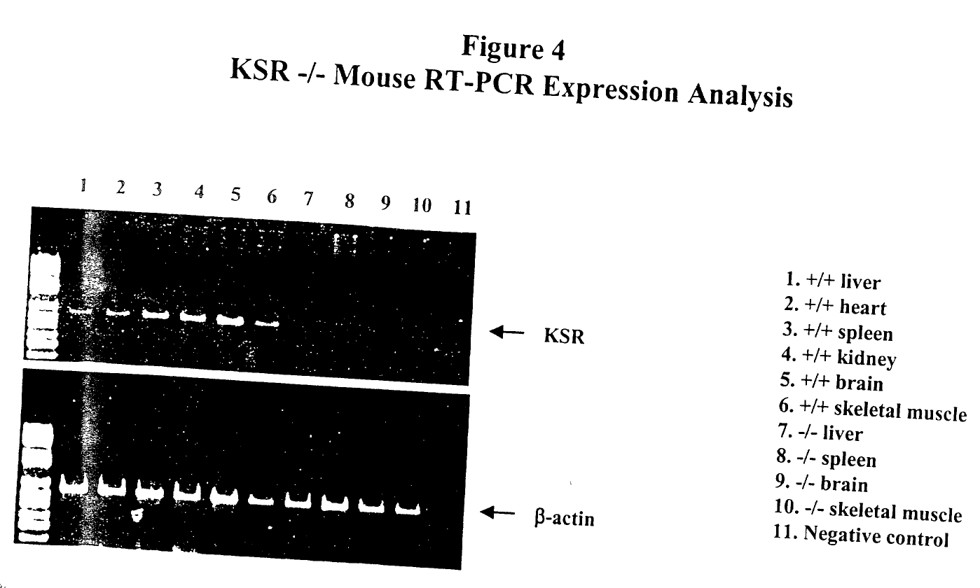

Total RNA was isolated using TRIzol® reagent (Gibco BRL, Rockville, Md.) from liver, heart, spleen, kidney, brain, and skeletal muscle of KSR+/+ mice and from liver, spleen, brain, skeletal muscle of KSR−/− mice. First strand cDNA was prepared on these RNA samples using the Superscript™ Preamplification System kit (Gibco BRL). PCR was done on these reverse transcriptase (RT) reactions using an oligo set specific for the KSR cDNA, which generated a 536 bp fragment (FIG. 4).

| | | |
|---|---|---|
| KSR-259F | 5′ GTGCTCAGTGTCTAACGACC 3′ | (SEQ ID NO:7) |
| KSR-795R | 5′ GATACAGGAGTCCGAGAGGC 3′ | (SEQ ID NO:8) |

The KSR-259F oligo is located in an exon upstream of the deleted region while the KSR-795R oligo is contained within an exon that was deleted upon homologous recombination. As a positive control for RNA quality, PCR was also conducted on the KSR cDNA using an oligo set specific for mouse β-actin (Clontech, Palo Alto, Calif.).

No tissues from the knockout mice demonstrated KSR expression by RT-PCR, although all knockout tissues were positive for β-actin. All wild-type control tissues were positive for KSR and β-actin expression by RT-PCR. As an additional control to check for genomic DNA contamination, reverse transcriptase controls were conducted on all test samples and all were negative (FIG. 4).

1. A genetically-modified, non-human mammal, wherein the modification results in a functionally disrupted KSR gene.

2. The mammal of claim 1, wherein said mammal is heterozygous for said modification.

3. The mammal of claim 1, wherein said mammal is homozygous for said modification.

4. The mammal of claim 1, wherein said mammal is a rodent.

5. The rodent of claim 4, wherein said rodent is a mouse.

6. A genetically-modified animal cell, wherein the modification comprises a functionally disrupted KSR gene.

7. The animal cell of claim 6, wherein said cell is heterozygous for said modification.

8. The animal cell of claim 6, wherein said cell is homozygous for said modification.

9. The animal cell of claim 6, wherein said cell is an embryonic stem (ES) cell or an ES-like cell.

10. The animal cell of claim 6, wherein said cell is human.

11. The animal cell of claim 6, wherein said cell is murine.

12. A method of identifying a gene that demonstrates modified expression as a result of reduced KSR activity in an animal cell, said method comprising assessing the expression profile of an animal cell containing a genetic modification that disrupts a KSR gene, and comparing said profile to that from a wild type cell.

13. The method of claim 12, wherein said cell is homozygous for a genetic modification that disrupts the KSR gene.


SEQUENCE LISTING (8 sequences)

SEQ ID NO: 1 - DNA, 24 bp, Homo sapiens
cctccttcttggctcaatctttcc

SEQ ID NO: 2 - DNA, 23 bp, Homo sapiens
tgctacttccatttgtcacgtcc

SEQ ID NO: 3 - DNA, 23 bp, Homo sapiens
gcaggatctcctgtcatctcacc

SEQ ID NO: 4 - DNA, 23 bp, Homo sapiens
gatgctcttcgtccagatcatcc

SEQ ID NO: 5 - DNA, 19 bp, Homo sapiens
caagctgaagccaccaagg

SEQ ID NO: 6 - DNA, 20 bp, Homo sapiens
acgtcgtcgattcggtttcc

SEQ ID NO: 7 - DNA, 20 bp, Homo sapiens
gtgctcagtgtctaacgacc

SEQ ID NO: 8 - DNA, 20 bp, Homo sapiens
gatacaggagtccgagaggc